(19) United States
(12) Patent Application Publication
(10) Pub. No.: US 2004/0265328 A1
(43) Pub. Date: Dec. 30, 2004

(54) Pathogenic and commensal vaccine antigens

(76) Inventors: Andrew Robinson, Salisbury (GB); Andrew Richard Gorringe, Salisbury (GB); Michael John Hudson, Salisbury (GB); Philippa Bracegirdle, Salisbury (GB); David McKay West, Salisbury (GB); Kerry Jane Oliver, Salisbury (GB); John Simon Kroll, London (GB); Paul Richard Langford, London (GB)

Correspondence Address: LESLIE MEYER-LEON, ESQ., IP LEGAL STRATEGIES GROUP P.C., 1480 FALMOUTH ROAD, P.O. BOX 1210, CENTERVILLE, MA, 02632-1210, US

(21) Appl. No.: 10/472,260

(22) PCT Filed: Mar. 22, 2002
(86) PCT No.: PCT/GB02/01399

(30) Foreign Application Priority Data
Mar. 22, 2001 (GB) 0107219.8

(51) Int. Cl. G01N 33/554; G01N 33/569; A61K 48/00; A61K 39/02; C12Q 1/68

(52) U.S. Cl. 424/190.1; 435/7.32; 514/44; 435/6

(57) ABSTRACT

The invention provides methods of screening commensal and pathogenic bacteria for previously unidentified vaccine antigens, based upon identifying polypeptide antigens that bind to sera raised against commensal bacterial proteins. Also provided are vaccine compositions and methods of preparing vaccine compositions comprising the antigens identified by the screening methods. Antigens and uses thereof are also described.

The present invention relates to compositions and methods for preparing vaccines that stimulate an immune response and are useful for prevention of neisserial infection. In particular, the present invention relates to vaccines that provide broad spectrum protective immunity.

Meningococcal disease is of particular importance as a worldwide health problem and in many countries the incidence of infection is increasing. Neisseria meningitidis (the meningococcus) is the organism that causes the disease, including meningococcal septicaemia, which is associated with rapid onset and high mortality, with around 22% of cases proving fatal.

At present, vaccines directed at providing protective immunity against meningococcal disease provide only limited protection because of the many different strains of N. meningitidis. Vaccines based upon the serogroup antigens, the capsular polysaccharides, offer only short lived protection against infection and do not protect against many strains commonly found in North America and Europe. A further drawback of these vaccines is that they provide low levels of protection for children under the age of 2 years, one of the most vulnerable groups that are commonly susceptible to infection. Newer conjugate vaccines now in use in the UK will address some of these problems but will only be effective against the C serogroup of the meningococcus

Gold et al. (Journal of Infectious Diseases, volume 137, no. 2, February 1978, pages 112-121) have reported that carriage of N. lactamica may assist in the development of natural immunity to N. meningitidis by induction of cross-reactive antibodies. This conclusion was based on the observation of cross-reacting antibodies having complement-dependent bactericidal activity produced in response to N. lactamica infection. However, Cann and Rogers (J. Med. Microbiol., volume 30, 1989, pages 23-30) detected antibodies to common antigens of pathogenic and commensal Neisseria species, but observed also that antibody to the same antigens was present in both bactericidal and non-bactericidal sera. Thus, it was not possible to identify the antigens responsible for cross-reactive bactericidal antibodies.

Live attenuated vaccines for meningococcal disease have been suggested by Tang et al. (Vaccine 17, 1999, pages 114-117) in which a live, attenuated strain of N. meningitidis could be delivered mucosally. Tang also commented on the use of commensal bacteria to protect against infection by pathogenic bacteria, concluding that the cross-reactive epitopes that induce protection against meningococcal infection have not been defined, and therefore that use of genetically modified strains of N. meningitidis would be preferred.

Frasch (Meningococcal Disease, Cartwright Ed. (1995); Ch. 10 , pp245-283) comprehensively describes the history and development of meningococcal vaccines. Frasch mentions the development of polysaccharide based vaccines, and also mentions the contemporary developments in the search for alternative vaccine candidates. Frasch deals in extensive sections on vaccines based on capsular components, such as serogroup A and serogroup C polysaccharide—note that, unlike pathogenic Neisseria, the commensal N. lactamica does not possess a capsule. It is known that capsules are often poorly immunogenic, but can be rendered immunogenic using carrier proteins.

Pollard (Pediatr. Infect. Dis. J. (2000); 19, pp. 333-45) provides an outline of the microbiology of the Gram negative N. meningitidis organism. Pollard identifies no fewer than twelve different serogroups based upon the chemical composition of the polysaccharide capsule that surrounds the outer membrane of the meningococcus—thus vaccines are targeted at these polysaccharide serogroups.

Cartwright et al (Vaccine 17 (1999), pp2612-2619) describes use of an alternative approach to vaccines, in which a novel vaccine composition comprises the PorA protein, and evokes a good immune response to strains of N. meningitidis. First, a specific surface-exposed protein is identified and OMVs that contain six PorA proteins are prepared.

Fusco et al (JID (1997); 175, pp. 364-72) describes the production of a group B meningococcal conjugate vaccine that includes purified recombinant PorB porin protein. Fusco et a/ teaches the identification of a surface protein from a pathogenic strain of Neisseria and the inclusion in a vaccine composition.

Perrin et al (11^{th} International Pathogenic Neisseria Conference, abstract (1998)), page 348 and Infect. Immun. (1999) describe the technique of genomic subtraction—i.e. subtracting the genomic data of commensal bacteria from pathogenic bacteria—which technique has the potential to identify regions of the chromosome likely to be involved in differential virulence of bacterial pathogens and thus likely to be potential vaccine antigens.

Pizza et al (Science (2000) 287; pp. 1816-1820) describes the whole genome sequencing of a virulent serogroup B strain of N. meningitidis in order to identify potential vaccine candidate antigens. Pizza et al utilises in silico prediction of surface expressed proteins and high through-put screening in order to identify suitable vaccine antigens from pathogenic Neisseria.

Nevertheless, there remains a need for further and better vaccines against neisserial infection.

It is desirable to identify immunogenic proteins for incorporation into a vaccine that gives protective immunity to infection from bacteria, especially pathogenic bacteria selected from the Neisseriaceae/Pasteurellaceae family of Gram negative bacteria—for example, N. meningitidis and N. gonorrhoeae. It further is desirable to provide a vaccine that confers protective immunity to infants as well as adults and whose protection is long term. It may also be of advantage to provide a vaccine that protects against sub-clinical infection, i.e. where symptoms of meningococcal or gonococcal infection are not immediately apparent and the infected individual may act as a carrier of the pathogen. It would further be of advantage to protect against all or a wide range of strains of N. meningitidis. It is still further desirable to provide a vaccine against other neisserial infection, notably gonorrhoea.

It is an object of the present invention to provide antigens, immunogens, compositions containing immunostimulating components, vaccines based thereon, and methods of identifying antigens that meet or at least ameliorate the disadvantages in the art.

The invention is based upon a new approach to the problems identified, aiming to identify immunogenic components in both commensal and pathogenic organisms of the same family as the pathogen against which the vaccines are to be protective.

The invention uses combined strategies for identifying antigens that interact with sera raised against commensal bacteria such as commensal Neisseria.

A first strategy involves the construction of a genomic library from a commensal bacteria, such as N. lactamica. The approach includes expressing fragments of the N. lactamica genome in recombinant phage, so as to create a phage display library where potentially antigenic polypeptides are expressed on the phage surface. Those phages that react with sera raised against a protective N. lactamica extract are isolated. N. lactamica sequences that code for the immunoreactive proteins are thereby identified as potential antigens, suitable for inclusion in vaccines to protect against neisserial infections.

The N. lactamica immunogenic protein antigens identified by the methods of the invention also serve as a starting point for identifying homologous proteins in other pathogenic bacteria, such as N. meningitidis. Thus, the invention allows for the identification of an entirely new class of vaccine antigens in both the commensal and pathogenic organisms.

A second strategy involves combining the sera raised against the commensal organism with preparations of antigens from pathogenic Neisseria. The binding between the antibodies in the commensal sera and the pathogen antigens is analysed to identify antigens with previously unknown immunogenic potential.

Accordingly, a first aspect of the invention lies in a method for identifying an antigen comprising:

a. obtaining antibodies against a commensal bacteria, or an extract from a commensal bacteria;

b. contacting the antibodies with one or more polypeptides obtained from either a commensal or a pathogenic bacteria;

c. determining whether the one or more polypeptides bind to antibodies; and

d. where a polypeptide binds to an antibody, identifying that polypeptide as an antigen.

In one embodiment of the invention the method comprises the steps of:

a. obtaining antibodies against a commensal bacteria or an extract from a commensal bacteria;

b. contacting the antibodies with one or more polypeptides obtained from an expression library of either a commensal bacteria or a pathogenic bacteria;

c. determining whether one or more polypeptides bind to antibodies;

d. where a polypeptide binds to an antibody, identifying that polypeptide as an antigen; and

e. isolating a clone from the expression library that expresses the antigen.

Antibodies against commensal bacteria or an extract from commensal bacterial may be contained within sera raised against the bacteria or the extract, and in specific examples below, antibodies are obtained by immunising an animal with commensal proteins. Antibodies may also be obtained from a patient infected with a commensal bacteria, from patient sera, from mucosal secretions or otherwise.

In a specific embodiment of the invention the isolation step (step (e)) comprises:

(i) identifying the molecular weight of the polypeptide that binds to the antibody in the sera;

(ii) correlating the molecular weight with the molecular weights of polypeptides encoded by the genome of the bacteria from which the polypeptide is derived; and

(iii) determining an identity for the polypeptide and the corresponding nucleic acid that encodes the polypeptide.

A number of methods are suitable for determining the molecular weight of the polypeptide. Suitable methods of molecular weight determination include mass spectrometry, electrophoresis or chromatography. In a preferred embodiment of the invention, discussed in detail below, the molecular weight of the polypeptide is determined via SELDI mass spectrometry.

The antigens are obtained from either a commensal organism or a pathogenic organism. Generally, the antigens are polypeptides and it is optional whether the polypeptides are in the form of proteins obtained from an expression library of the relevant organism, or whether they are in the form of a cell extract. For mass spectrometry based embodiments of the present invention, it is preferred that the polypeptides be in the form of a solution or suspension, typically a detergent extract of outer membrane proteins. For the genomic library based embodiments, polypeptides are preferably expressed from a genomic library such as a phage display library. In the latter case, a clone that expresses the polypeptide antigen will be located within a phagemid vector.

The genomic libraries suitable for use in the methods of the invention can be derived from either commensal or pathogenic genomes. If a pathogenic genome is used then the results of the screening steps will be to identify those polypeptides that have cross reactivity between pathogenic and commensal organisms.

Commensal micro-organisms are those that can colonize a host organism without causing disease. A number of different commensal bacteria exist. Commensal Neisseria are suitable for use in the invention, and these commensal Neisseria are typically selected from the group consisting of N. lactamica, N. cinerea, N. elongata, N. flavescens, N. polysaccharea, N. sicca, N. perflava and N. subflava. Different species of these commensal organisms are known to colonise the buccal or nasal areas or other mucosal surfaces and hence each species may generate different antigens according to the area of the body it normally colonises.

Sera raised against commensal organisms have been found to be particularly advantageous as a starting point for the screening methods of the present invention. Unlike sera raised against pathogenic organisms or extracts (e.g. convalescent sera), commensal sera tends to react with a broader range of antigens. Sera raised against pathogenic organisms or extracts of such organisms tends to demonstrate an immunoreactive bias towards certain dominant antigens. For example, sera raised in rabbits against an outer membrane protein preparation from N. meningitidis are biased towards immunodominant antigens such as PorA. As a result, a significant disadvantage of using such sera is that the immunodominant antigens also tend to be the antigens that demonstrate greatest variability across the strain. Hence vaccines derived from sera raised against pathogens such as N. meningitidis tend to have poor cross-reactivity between strains, thus affording lower levels of protection.

Immunodominant antigen bias is seen to a much lesser extent in sera raised according to the invention against commensals such as N. lactamica, where there are far fewer immunodominant antigens. As a result sera raised against commensal organisms provides an ideal basis for identifying potential vaccine antigens that demonstrate less variability between strains and allow for the production of vaccines that provide broader spectrum, and longer term protection.

Where the method of the invention is used to first identify a clone/polypeptide from a commensal bacteria, a further method step can optionally be performed comprising the steps of:

(i) using the nucleic acid sequence of the isolated clone encoding the polypeptide antigen from the commensal bacteria to identify homologous sequences in pathogenic bacteria; and

(ii) cloning the homologous sequences from the pathogenic bacteria in order to generate the equivalent pathogenic bacterial polypeptide antigen.

Hence, both the commensal protein antigen and the corresponding pathogen protein antigen can be identified. This allows for further analysis of potential antigenic regions of the homologous polypeptides and also the design of fusion proteins containing pathogenic and commensal sequences with greater vaccine antigenic potential.

The sera used in the present invention is typically raised in mice or rabbit hosts which are exposed to commensal proteins. The sera are suitably raised against a preparation of outer membrane proteins obtained via standard detergent extraction protocols. Alternatively, whole cells of commensal bacteria can be injected into the host animal. The results of the screening steps can also be biased by choosing particular cell extracts against which the sera is raised. For example, an outer membrane protein extract of a specified molecular weight range could be used in order to generate sera with a particular immunoreactivity profile.

The sera used in the polypeptide antigen screening steps can also be purified. Typically, the IgG component of the sera is isolated, especially when the mass spectrometry embodiments of the invention are to be used.

A second aspect of the invention provides a method for identifying an antigen, suitable for inclusion in a vaccine composition, comprising the steps of:

(a) obtaining sera raised against an outer membrane protein extract of N. lactamica;

(b) contacting the sera with an N. lactamica phage display library, preferably one comprising the entire N. lactamica genome;

(c) identifying a phage that tests positive for a binding interaction with the sera, and isolating the positive phage;

(d) extracting phagemid vector from the positive phage and characterising the cloned N. lactamica genomic sequence located therein; and

(e) determining the polypeptide encoded by the N. lactamica genomic sequence and identifying the polypeptide as an antigen

In a preferred embodiment the method of the invention further comprises the step of:

(f) comparing the sequence of the N. lactamica polypeptide antigen with an N. meningitidis genome sequence in order to identify a N. meningitidis homologue polypeptide antigen.

A third aspect of the invention provides a method for identifying an antigen, suitable for inclusion in a vaccine composition, comprising the steps of:

(a) obtaining sera raised against an outer membrane protein extract of N. lactamica;

(b) isolating the IgG component of the sera;

(c) binding the isolated IgG to a solid phase;

(d) contacting the bound IgG with polypeptides obtained from an extract of N. meningitidis cells;

(e) isolating solid phase-IgG-polypeptide complexes that are formed by the binding of polypeptides to IgG;

(f) analysing solid phase-IgG-polypeptide complexes via SELDI mass spectrometry;

(g) correlating molecular weights obtained for the polypeptide from (f) with molecular weights of known and putative polypeptides from the N. meningitidis genome database; and

(h) identifying as antigens those N. meningitidis polypeptides encoded by genes determined from the correlated molecular weights of (g).

Further aspects of the invention provide methods of preparing vaccine compositions comprising identifying one or more antigens according to the methods described above, and combining the antigen(s) with a pharmaceutically acceptable carrier. A pharmaceutically acceptable carrier suitable for use in the composition is, for example, aluminium hydroxide although any carrier suitable for oral, intravenous, subcutaneous, intraperitoneal or any other route of administration is suitable.

The antigens from commensal or pathogenic Neisseria are suitably administered in vaccine compositions, either as whole cells, preparations of outer membrane vesicles (OMVs) from whole cells, or in recombinant form. Where whole cells or OMVs are administered, the invention provides for a method of increasing the antigenic potential of a commensal Neisseria, by introducing or up-regulating the expression of cross-reactive antigens in the whole cell, typically via introduction of gene constructs enabling recombinant production of further antigenic components.

Known methods of OMV isolation, such as by deoxycholate treatment, are suitable for preparation of compositions of the invention.

Formulations of the composition of the present invention with conventional carriers or adjuvants provide a composition for treatment of infection by pathogenic bacteria, such as those from the Neisseriaceae/Pasteurellaceae family of Gram negative bacteria.

Hence, the use of a composition of the invention can result in stimulation or production of protective antibodies in the recipient de novo or if the individual has already been colonised by a commensal or pathogenic bacterium, may result in an enhancement of naturally-existing antibodies.

Antigens identified by the methods of the present invention may be suitably included in vaccine compositions intended to provide protective immunity to pathogenic bacterial infection. Compositions, comprising an antigen with a pharmaceutically acceptable carrier can include antigens that are polypeptides comprising at least 10 contiguous amino acids encoded by any of the nucleotide sequences identified herein and discussed in more detail below.

The term “antigen” as used herein refers to both proteinaceous and non-proteinaceous antigens, and when proteinaceous includes proteins, polypeptides, oligopeptides of at least 10 contiguous amino acids, as well as fragments of proteins and polypeptides. It should be noted that antigen fragments can be expressed from part of a nucleic acid sequence that encodes a full length protein, or can be derived from the enzymatic cleavage of a full length polypeptide. In the latter case, an antigen that is a fragment obtained via proteolytic enzyme digestion can retain aspects of tertiary structure essential to retention of antigenicity.

A “vaccine antigen” is an antigen that when included in a vaccine composition elicits protective immunity to bacterial infection.

The vaccine compositions of the present invention are particularly suited to vaccination against infection of an animal. The term “infection” as used herein is intended to include the proliferation of a pathogenic organism within and/or on the tissues of a host organism. Such pathogenic organisms typically include bacteria, viruses, fungi and protozoans, although growth of any microbe within and/or on the tissues of an organism are considered to fall within the term “infection”.

In the process of performing the screening methods of the invention a number of useful vaccine antigens have been identified. The screening methods utilised sera raised against an outer membrane extract from the commensal N. lactamica . A number of N. lactamica and N. meningitidis nucleic acid sequences encoding potential vaccine antigens have been identified. Some of the identified N. meningitidis sequences are known vaccine antigens, although the vast majority of sequences identified encode proteins with previously unknown vaccine potential.

The present inventors have also constructed a novel N. lactamica genomic library in lambda phage. The N. lactamica nucleic acid sequences identified via the methods of the invention thus represent novel gene sequences that when expressed provide novel polypeptides, previously uncharacterised and not before isolated from N. lactamica. These polypeptides show significant utility as vaccine antigens.

Accordingly, further aspects of the invention provide for polypeptides encoded by all or a part of a N. lactamica nucleic acid sequence selected from the group consisting of SEQ. ID NOS: 1; 5; 11; 15; 19; 23; 27; 31; 35; 39; 43; 47; 51; 55; 59; 63; 67; 71; 75; 79; 83; 87; 91; 95; and 99. Polypeptide antigens derived from the polypeptide expressed from these nucleic acid sequences can be suitably included in vaccine compositions that protect against meningococcal disease.

The invention also provides for polypeptide antigen expressed from a nucleic acid sequence having at least 80% homology, preferably at least 80% similarity, or at least 90% homology, preferably at least 90% similarity with a nucleic acid sequence selected from the N. lactamica or N. meningitidis sequences of the invention.

A number of sequence comparison algorithms are known in the art suitable for determining homology between nucleic acid (or polypeptide) sequences. These algorithms are suitable for both sequence comparison and analysis of nucleic and amino acid sequences. Software and systems utilising algorithms such as BLAST, FASTA, Tepitope™, PepTool™ and EpiMer ™ are suitable for use in the methods of the present invention. In use, the skilled person is able to readily identify areas of homology between two or more sequences. Further, these algorithms facilitate sequence analysis to the extent that particular regions or localised domains of high sequence identity can be pinpointed and thus potential sub-domain antigens can be identified. The sequence analysis thereby enables the identification of localised antigenic regions that can be included in vaccine compositions of the invention. The method of the invention is particularly useful where inclusion of the whole protein encompassing a desired antigenic region would be deleterious, possibly due to auto-immune responses that might be caused in a host organism, or due to the presence of masking domains that would hide the antigenic region from the host immune system.

In still further aspects of the present invention an isolated N. lactamica nucleic acid molecule is provided, selected from the group consisting of SEQ. ID NOS: 1; 5; 11; 15; 19; 23; 27; 31; 35; 39; 43; 47; 51; 55; 59; 63; 67; 71; 75; 79; 83; 87; 91; 95; and 99. Also provided are vectors comprising the isolated nucleic acid molecules.

The corresponding polypeptides translated from the N. lactamica sequences of the invention are also provided herein in SEQ. ID NOS: 2; 6-8; 12; 16; 20; 24; 28; 32; 36; 40; 44; 48; 52; 56; 60; 64; 68; 72; 76; 80; 84; 88; 92 and 100. These polypeptides and parts thereof, are useful as vaccine antigens.

The terms “a part of” or “a fragment of” as used herein is intended to refer to parts of the polypeptide antigen that demonstrate an antigenicity that is equivalent to that of the entire protein itself. In essence, an antigenic motif or domain that consists of, for example, only 20% of the whole protein can have an equivalent value as a vaccine antigen as the full length protein. A part or fragment of a full length polypeptide antigen will typically comprise around 10 or more contiguous amino acid residues of that full length antigen, although in certain cases fewer than 10 residues might be sufficient to generate some protective immunity.

As mentioned previously the screening methods of the present invention have also succeeded in identifying a number of N. meningitidis proteins as candidate vaccine antigens. The nucleic acid sequences that encode these polypeptide antigens are shown in SEQ. ID NOS: 3; 9; 13; 17; 21; 25; 29; 33; 37; 41; 45; 49; 53; 57; 61; 65; 69; 73; 77; 81; 85; 89; 93; 101; 103; 105; 107; 109; 111; 113; 115; 117; 119; 121; 123; 125; 127; 129; 131; 133; 135; 137; 139; 141; 145; 147; 149; 151; 153; 155; 157; 159; 161; 163; 165; 167; 169; 171; 173; 175; 177; 179; 181; 183; 185; 187; 189; 191; 193; 195; and 198. The corresponding polypeptide translations are provided in SEQ. ID NOS: 4; 10; 14; 18; 22; 26; 30; 34; 38; 42; 46; 50; 54; 58; 62; 66; 70; 74; 78; 82; 86; 90; 94; 102; 104; 106; 108; 110; 112; 114; 116; 118; 120; 122; 124; 126; 128; 130; 132; 134; 136; 138; 140; 142-144; 146; 148; 150; 152; 154; 156; 158; 160; 162; 164; 166; 168; 170; 172; 174; 176; 178; 180; 182; 184; 186; 188; 190; 192; 194; 196-197; and 199.

Hence, further aspects of the invention provide for vaccine compositions comprising the newly identified N. meningitidis polypeptide antigens or parts thereof.

Yet further aspects of the invention provide for uses of the polypeptides expressed from the nucleic acid sequences, or the specified polypeptide sequences themselves, as vaccine antigens. Further uses include the use of the polypeptides expressed from the nucleic acid sequences, or the specified polypeptide sequences themselves in the manufacture of medicaments for vaccination against meningococcal disease.

Another aspect of the invention provides for a method of preparing a composition for vaccination against infection by pathogenic bacteria, comprising:

(1) obtaining a first antigen from a commensal Neisseria;

(2) (a) comparing the amino acid sequence of the first antigen with the amino acid sequence of a second antigen from a pathogenic bacteria or, (b) comparing the sequence of a nucleic acid which codes for the first antigen with the sequence of a nucleic acid that codes for the second antigen; and, if the first antigen is homologous to the second antigen or if the nucleic acid sequence for the first antigen is homologous to the nucleic acid sequence for the second antigen, then

(3) preparing a composition for vaccination against bacterial infection comprising the first antigen

The antigen from a commensal Neisseria or nucleic acid sequence from a commensal Neisseria can be compared with a library of antigens or nucleotide sequences from a pathogenic bacteria, to determine whether there is a corresponding homologous antigen from the pathogenic bacteria.

The terms “homologous” and “homology” and related terms as used herein mean that the immune response to the commensal antigen cross-reacts with a pathogen. Such homology is present if an immune response to the commensal antigen is protective against challenge by a pathogen. Such homology is also present if there is sequence similarity, e.g. sequence homology between the respective commensal and pathogen sequences of at least 50%, preferably at least 70%, more preferably at least 80%. The sequences are either amino acid sequences or nucleic acid sequences that encode amino acid sequences. The level of similarity can be either the level of identity between the entirety of the sequences, the level of identity between a portion of the sequences or the similarity in antigenic equivalence. It is apparent that the level of identity between the sequences is a function of the primary structure of the amino acid sequences, whereas the level of antigenic equivalence/homology is a function of the secondary and tertiary structure of the amino acid sequences.

In determining the level of homology between the amino acid or nucleic acid sequences for the antigenic component in the commensal Neisseria, and that of the antigenic component in the pathogenic bacteria, the level of homology is determined with respect to significant antigenic epitope, domains or subunits and is not limited to an overall level of homology.

For example, if there is a high level of homology to the sequence of a particular subunit of a membrane associated surface protein, but low levels of homology to the associated membrane spanning domain and intracellular regions, then the level of effective antigenic homology will still be considered high and useful in the invention even though the overall level of sequence homology is low.

It is important to note that the antigenic components of the compositions of the invention are preferably amino acid sequences that are immuno-apparent, or “visible”, to the immune system of a host organism. Thus, a 50% homology between the immuno-apparent regions of a protein may not correspond to a high overall homology between the sequences of the commensal and pathogenic versions. Indeed, identifying specific domains of high sequence homology between antigenic components from commensal and pathogenic species is sufficient to identify an antigen from a commensal species that is suitably included in a vaccine composition that provides protective immunity to infection from the pathogenic species.

Ideally there is a high level of homology, in excess of 90 percent, though above 50 percent is usually sufficient, preferably above 70 percent.

The method of the invention comprises identifying an antigen from a commensal Neisseria that is homologous in amino acid sequence to a sequence from a pathogenic bacteria. Preferably the pathogenic bacteria is a Gram negative bacteria, more preferably a pathogenic Neisseria, for example N. meningitidis or N. gonorrhoeae. Alternatively, the bacteria is selected from pathogenic members of the Neisseriaceae (includes Neisseria, Branhamella, Moraxella, Acinetobacter, Kingella) and the Pasteurellaceae (Pasteurella, Haemophilus, Actinobacillus) families of Gram negative bacteria. These micro-organisms are characterised by the ability to inhabit mucosal surfaces in a host organism and to cause infections such as otitis media (middle ear infection). Due to the fact that they tend to inhabit a similar environmental niche and are often co-exist on the same mucosal surface, it can be difficult to discriminate clinically between the pathogenic members of this subgroup of bacteria.

Commensal micro-organisms are those that can colonize a host organism without causing disease. A number of different commensal Neisseria are suitable for use in the invention, and these commensal Neisseria may be selected from the group consisting of N. lactamica, N. cinerea, N. elongata, N. flavescens, N. polysaccharea, N. sicca, N. perflava and N. subflava. Different species of these commensal organisms are known to colonise the buccal or nasal areas or other mucosal surfaces and hence each species may be administered according to the known area of the body it normally colonises. Hence also, use of a composition of the invention may result in stimulation of production of protective antibodies de novo or if the individual has already been colonised to a certain extent may result in an enhancement of naturally-existing antibodies.

The Neisseria meningitidis (serogroups A and B), Haemophilus influenzae and Pasteurella multocida (PM70) genomes have been sequenced and published. The genomic data is available from the Sanger Institute (Cambridge, UK) or on the internet (www.sanger.ac.uk, www.ebi.ac.uk/genomes and www.tigr.org) and the number of fully sequenced bacterial genomes available is anticipated to increase dramatically in the future. Hence it is possible for the neisserial commensal nucleic acid sequence to be compared with a genome sequence of a plurality of pathogenic bacteria.

The antigens from a commensal Neisseria identified in the method of the present invention need not be limited to surface visible antigens. Indeed, it is a surprising observation that a number cytoplasmic and endosomal proteins previously thought not to be visible to the host immune system do have antigenic potential. Thus, it is of considerable advantage that the present invention allows for the inclusion of a broader range of antigenic components in vaccine compositions than was previously thought possible. As apparent from the literature discussed in the background section above, a large number of candidate antigens have been identified in pathogenic Neisseria and have been or are being tested for their value in vaccines. A further optional screening step in the invention is to retain antigen from a commensal Neisseria that corresponds to antigens already identified from studies on pathogens as having actual or potential value.

Candidate antigens identified in commensal Neisseria by the method of the invention are evaluated in a number of ways. Some candidate antigens have sequence homologous to conserved sequence in a plurality of different pathogenic species and thus demonstrate the potential for broad spectrum protection. Other candidate antigens demonstrate a high level of homology to a sequence in a single pathogenic species, and thus demonstrate the potential for strong antigenic activity with respect to this single species of pathogen.

Candidate antigens are also evaluated on the basis of their suitability for inclusion in vaccine compositions of the invention. Some candidate antigens are more readily incorporated in outer membrane vesicles (e.g. membrane associated proteins) than others and are therefore selected for this particular mode of delivery.

In one particular method, a protein is identified in the commensal Neisseria. This protein is then screened for reactivity with an antibody preparation which is known to bind to the commensal. For example, the antibody preparation can be prepared using an extract of commensal membrane. If the screen is positive, that is to say if the protein is recognized by the antibody preparation, then it is identified as an antigen. This first screen thus confirms that the protein is antigenic and is likely to be expressed on the surface of the commensal.

A second screening step is to investigate if there is a corresponding antigenic sequence in a pathogenic Neisseria, and this is suitably done as described above. If this second screen is positive then third and further screens, to identify most preferred vaccine candidates include selection by size, selection by frequency of existence of a corresponding antigen in all pathogenic species.

Thus in an example of the invention in use, a detergent extract of a commensal Neisseria (e.g. N. lactamica) is used to vaccinate mice. Mice are subjected to challenge from a pathogenic member of the Neisseriaceae/Pasteurellaceae family (e.g. N. meningitidis, Moraxella catarrhalis, Pasteurella multocida or Haemophilus influenzae). Convalescent sera is obtained from mice that survive the challenge, and is used to screen an expression library from the commensal Neisseria to identify candidate antigens. The nucleotide and amino acid sequence for the identified candidate antigens can then be determined and compared by sequence similarity and other analysis for homology to sequences from the pathogenic organism. Candidate antigens are selected for their suitability and included in vaccine compositions.

In an example of the invention in use antigens from commensal Neisseria can be evaluated for their suitability for inclusion in a vaccine, by the following steps of:—

obtaining an amino acid sequence for an antigen from a commensal

Neisseria, or obtaining a nucleotide sequence encoding the antigen;

comparing the sequence obtained with a corresponding sequence from a pathogenic bacteria; and

identifying an antigen in which the total sequence homology exceeds 50%.

A composition for vaccination against neisserial infection is then prepared by identifying an antigen according to this method and incorporating said antigen into the composition.

This latter aspect of the present invention thus represents a departure from previously held wisdom in the area of vaccine preparation. According to the invention, vaccine antigens are identified starting with an antigen expressed in a commensal species. This antigen is suitably tested to determine whether it is expressed on the surface of the commensal and if so it is investigated whether a corresponding protein, that is to say a protein which is at least 50% homologous, is present in the pathogen. When the corresponding protein is identified in the pathogen, the vaccine is then based upon either the commensal protein, or an immunogenic fragment thereof, or from the pathogenic species. The invention thus differs substantially from prior approaches to obtaining vaccines in which subtraction work was used to identify antigens seen only in the pathogen.

An advantage of the current approach is that handling the commensal organism carries fewer risks during preparation of the vaccines. A further advantage is that antigens in commensals tend to demonstrate fewer intra-species variations. Thus, the commensal-derived antigens can offer a broader spectrum of immunity, albeit in some circumstances of a level of protection that is lower against certain pathogenic strains than an antigen derived from that particular pathogenic species. A benefit is that the commensal-derived antigen generally possesses at least a low level of protection against a wider range of strains.

According to the invention, therefore, a trade-off is accepted between potency against individual strains in favour of cross-reactivity against many strains. In practice, vaccination programs are crude in that all individuals in, say, a given population such as within one country tend to be administered the same vaccine, regardless of whether in particular parts of the country one pathogenic strain in more prevalent than another. Cross-reactivity of antigenic component as provided in the instant invention ensures at least a base-line of protection for the vast majority of those vaccinated rather than high protection in some and the risk of absence of protection in others.

In examples of the invention described below in more detail, the antigen is a fragment of a commensal Neisseria protein. Antibodies raised against a commensal Neisseria detergent extract are used to identify the antigen. This antigen, which as mentioned is a fragment of an intact commensal protein, is used to identify a corresponding protein having a minimum level of homology in the pathogenic organism. The existence of a pathogenic partner to the antigen from a commensal Neisseria marks both the commensal antigen and the pathogenic antigen as a vaccine candidates. As mentioned previously, due to the lack of Immunodominant antigens in sera raised against commensal proteins, there exists the real opportunity to identify novel pathogenic antigens also.

In an example described in more detail below, a genomic library was prepared from Nasseiria lactamica. Genomic DNA was partially digested by Mbol. Digested DNA of between 1 and 4 kb was ligated into the ZAP Express vector (Stratagene) and packaged using the Gigapack III Gold packaging extract (Stratagene). Ligated and packaged DNA was plated and plaque lifts were performed. Plaques were screened using rabbit serum raised against an N. lactamica detergent extract previously identified as protective against meningococcal challenge in an experimental meningococcal infection model. Positive plaques, reacting with this N. lactamica serum were picked and purified. After ensuring that the inserts were of different sizes by PCR, the pBK-CMV phagemid vector was excised from the ZAP Express vector for each clone. The phagemids were purified and inserts were then sequenced using T3 and T7 primers. The sequences produced were compared with the meningococcal genome and the homologous meningococcal proteins are listed below. NMA numbers are the gene number assigned in the meningococcal serogroup A genome. NMB numbers are those assigned in the meningococcal serogroup B genome

The invention also provides for a quality control method of determining if a candidate antigen is present in a composition under test by:

(i) identifying the candidate antigen in the composition, or

(ii) identifying an immune response in a host animal exposed to the composition appropriate to the presence of the antigen in said composition.

Typically, a preparation to be evaluated comprises a number of different antigenic components as is the case for OMV-based vaccines. The method of the invention allows for the screening of these preparations to assess whether the candidate antigen(s) is present and present to an acceptable degree.

Recombinant candidate antigen is also utilised for quality control assays routinely in testing of sera from animals vaccinated with a composition comprising the antigen. If the sera from these animals reacts with the candidate antigen sufficiently then it is considered that the composition contains adequate amounts of the candidate antigen. If there is an insufficient reaction then the composition is considered to be defective. The reaction between sera and recombinant antigen is suitably mediated via a number of techniques commonly known in the art, for example, recombinant antigen can be placed on microparticles such that in the presence of sera containing the antibody to the antigen an agglutination reaction occurs. This assay can also be adapted to test the vaccine composition itself by replacing the recombinant antigen with the vaccine composition in the aforementioned steps.

A defective vaccine can be discarded if it is found not to contain a desired candidate antigen, or is found in protection tests not to evoke a desired immune response.

The invention is now described in more detail with references to the following examples.

### EXAMPLES

### Example 1

### Genomic Screen

1. Preparation of N. lactamica Genomic Phage Display Library

1.1. Preparation of N. lactamica Genomic DNA

100 ml MHB (Oxoid) was inoculated with a loopful of plate grown N. lactamica , strain Y92-1009 and incubated with shaking at 37° C. for 18 h. The culture was centrifuged at 4000 g and the pellet resuspended in 9.5 ml TE buffer (10 mM Tris, 1 mM ethylenediaminetetraacetic acid (EDTA), pH 8.0). 0.5 ml 10% (w/v) sodium dodecyl sulphate (SDS) and 50 ml 20 mg/ml proteinase K (Sigma) was added to the suspension and this was incubated for 1 hour at 37° C. 1.8 ml of 5 M NaCl and 1.5 ml 10% cetyltrimethylammoniumbromide (CTAB) in 0.7 M NaCl was added and the solution incubated at 65° C. for 20 min. DNA was extracted form the lysed cells by the addition of an equal volume of chloroform:isoamyl alcohol (Sigma). The solution was centrifuged at 6000 g for 10 min and 0.6 of the total volume of isopropanol was added to precipitate the DNA. Precipitated DNA was washed in 1 ml 70% (v/v) ethanol and recovered by centrifugation at 10000 g for 5 min, the supernatant discarded and the pellet resuspended in 4 ml TE buffer. 1.075 g/ml CsCI (Sigma) and 50 ml of 10 mg/ml ethidium bromide (Sigma) were added and the solution was centrifuged in quick-seal centrifuge tubes at 250000 g for 18 h at 15° C. The CsCI gradient was visualised under longwave UV and the band removed. Ethidium bromide was removed by sequential extractions with water saturated butanol. CsCl was removed by precipitation of the DNA with ethanol at 4° C. for 15 min followed by centrifugation at 10000 g for 15 min. The pellet was resuspended in TE buffer for long term storage.

1.2. Preparation of Partially Digested Genomic DNA

10 mg genomic DNA was digested as follows. 10 mg genomic DNA, 1 mg BSA, 10 ml NEBuffer 3 (New England Biolabs), 6 ml Mbol (New England Biolabs) and 63 ml molecular biology grade water (Sigma) were incubated at 37° C. for 2 h. The products were analysed on a 0.8% (w/v) low melting point agarose gel (Sigma). Bands of between 1 and 4 kb were located using longwave UV and cut from the gel. Digested DNA was removed from the gel using the QlAquick Gel Extraction Kit (Qiagen), following the protocol supplied. Extracted DNA was stored in TE buffer.

1.3. Ligation

To ligate partially digested DNA to the ZAP Express vector (Stratagene) a ligation reaction was set up as follows. 1 mg vector, 0.4 mg digested DNA, 0.5 ml 10×T4 DNA ligase buffer (New England Biolabs), 2.7 ml molecular biology grade water and 10U T4 DNA ligase (New England Biolabs) were incubated at 4° C. for 18 h.

1.4. Packaging

The phage particles were packaged using the Gigapack III Gold packaging extract (Stratagene). 3 ml ligation reaction was added to the packaging extract and mixed well. The mixture was centrifuged at 6000 g for 5 seconds and incubated at room temperature for 2 h. 500 ml SM buffer (5.8 g NaCl, 2 g MgSO₄.7H₂O, 50 ml 1 M Tris (pH 7.5), 5 ml 2% (w/v) gelatine diluted to 1 L with H₂O) and 20 ml chloroform were added to the mixture, the contents mixed, centrifuged briefly and the supernatant stored at 4° C.

1.5. Plating Packaged Ligation Product

E. coli, strain XL1 Blue MRF′, was grown overnight on an LB agar plate at 37° C. for 18 h. 10 ml LB broth supplemented with 10 mM MgSO₄ and 0.2% (w/v) maltose was inoculated with a single colony and incubated with shaking at 37° C. for 6 h. The cells were centrifuged at 1000 g for 10 minutes, the supernatant removed and the pellet resuspended in 10 mM MgSO₄. The OD₆₀₀ was adjusted to 0.5. 2 ml of the final packaged reaction was mixed with 200 ml XL1 Blue MRF′ and incubated with gentle shaking at 37° C. for 15 min. 3 ml NZY top agar, 15 ml 0.5 M isopropyl-b-D-thiogalactopyranoside (IPTG) and 12.5 mg 5-bromo-4-chloro-3-indolyl-b-D-galactopyranoside (X-gal) (Sigma) were added and the mixture poured immediately onto NZY agar plates and incubated at 30° C. for 18 h. 20 plates were prepared to cover the entire N. lactamica genome. Bacteriophage were recovered by overlaying the plates with 8-10 ml SM buffer and incubating with gentle shaking at 4° C. for 18 h. 5% (v/v) chloroform was added to the bacteriophage suspension and the cell debris removed by centrifugation at 500 g for 10 min. Chloroform (0.3% v/v) was added to the supernatant and the library was stored at 4° C.

2. Analysis of Genomic Library

2.1. Plaque Lifts

3 ml of the bacteriophage library was plated as described above on to enough NZY agar plates to cover the genome. After incubation at 30° C. for 18 h, IPTG soaked nitrocellulose membranes (Amersham Pharmacia Biotech) were applied to the plates and incubated at 30° C. for a further 18 h. The membranes were carefully removed and blocked with phosphate buffered saline (PBS) containing 0.05% (v/v) Tween-20 and 1% (w/v) milk powder (Marvel) for 1 h. The membranes were washed with PBS containing 0.05% Tween 20 (PBS-T) and then incubated with rabbit serum raised against N. lactamica detergent extracted OMPs diluted with PBS-T for 1 hour. After washing as before, the membranes were incubated with anti-rabbit horseradish peroxidase (HRP) (ICN) for 1 hour, washed with PBS and developed with 0.5 mg/ml 4-chloro-1-naphthol (Sigma) to identify cross reactive phage.

2.2. Plaque Purification

A plug of agar containing the positive plaque was removed from the plates for each plaque identified. The plugs were placed in 1 ml SM buffer containing 0.5% (v/v) chloroform and incubated at 4° C. for 18 h. 10 ml phage suspension was plated, lifted and positives identified as previously described. This was carried out for each positive and repeated until the suspensions were pure. Long term storage of phage was at 4° C. in SM buffer.

2.3. Polymerase Chain Reaction (PCR)

Positive phage were plated as previously described. One plaque was picked for each positive, pipetted into 500 ml molecular biology grade water, vortexed and incubated at 4° C. for 18 h to release phage particles from the agar. This was used as the template for PCR. The following reaction mixture was used to amplify N. lactamica inserts from positive phage; 1 ml T3 primer (Life Technologies), 1 ml T7 primer (Life Technologies), 1 ml template, 2.5 U Taq DNA polymerase (Roche), 5 ml 10×PCR buffer (Roche), 1 ml of 10 mM dNTP (Roche) and 40.5 ml were mixed on ice in a 200 ml PCR tube (Anachem-Scotlab) for each template. The reactions were heated to 94° C. for 3 min. Thermal cycling was repeated 35 times as follows; 94° C. for 30 seconds, 52° C. for 30 seconds, 72° C. for 2.5 min. The reactions were finally incubated at 72° C. for 10 min.

2.4. Phagemid Excision

Positive phage were plated as previously described. Individual phage stocks were prepared from the transfer of one plaque to 500 ml SM buffer containing 20 ml chloroform, which was then vortexed and incubated at 4° C. for 18 h. E. coli, strain XL1 Blue MRF′ was grown in 10 ml LB broth supplemented with 0.2% (w/v) maltose and 10 mM MgSO₄ at 30° C. for 18 h. The cells were centrifuged at 1000 g for 15 min and the pellet resupended to an OD₆₀₀ of 1 in 10 mM MgSO₄. 200 ml resupended XL1 Blue MRF′ was combined with 250 ml phage stock and 1 ml ExAssist helper phage (Stratagene) and incubated at 37° C. for 15 min. 3 ml NZY broth was added to each mixture and these were further incubated with gentle shaking at 37° C. for 2.5 h and finally heated to 70° C. for 20 min and centrifuged at 1000 g for 15 min. The supernatant, consisting of excised pBK-CMV phagemid vector, was decanted and stored at 4° C. This was repeated for each phage stock.

2.5. Plating Excised Phagemid

E. coli, strain XLOLR, was grown in 10 ml NZY broth at 30° C. for 18 h, centrifuged at 1000 g for 15 min and the pellet resuspended in 10 mM MgSO₄ to and OD₆₀₀ of 1. 100 ml of the phagemid supernatant was mixed with 200 ml resuspended cells and incubated at 37° C. for 15 min. 300 ml NZY broth was added and the mixture further incubated at 37° C. for 45 min. 200 ml of the cell mixture was plated on LB agar supplemented with 50 mg/ml kanamycin (Sigma) and incubated at 37° C. for 18 h.

2.6. Phagemid Purification

Phagemids were purified using Wizard Plus Minipreps (Promega) following protocol supplied.

2.7. Sequencing

The purified phagemid stocks were then sequenced using T3 and T7 primers. The sequences are shown followed by their translation products and the corresponding N. meningitidis homologues (nucleic acid and protein) in SEQ ID NOS: 1-102.

The N. lactamica proteins deduced from the genomic screen and thus identified as candidate vaccine antigens are shown in Table 1, below.

TABLE 1
N. lactamica genomic library: proteins deduced from sequences

| | | | Predicted |
|---|---|---|---|
| | | Swissprot | protein |
| SEQ ID | | Homologue | molecular |
| NO | N. meningitis homologue | code* | weight |
| 1 | Putative integral membrane protein | Q9JWY0 | 23.0 kDa |
| 5 | Putative ribonuclease (VacB) | Q9JUD1 | 60.4 kDa |
| 11 | Dihydrolipoamide acetyltransferase | Q9JU07 | |
| | (pyruvate dehydrogenase component) | | |
| 15 | Hypothetical protein | Q9JX69 | 11.6 kDa |
| 19 | Hypothetical protein | Q9JX70 | 7.0 kDa |
| 23 | Hypothetical protein | Q9JQT0 | 19.8 kDa |
| 27 | Hypothetical protein | Q9JX71 | |
| 31 | Hypothetical protein | Q9JYD0 | 38.3 kDa |
| 35 | Putative integral membrane protein | Q9JTB4 | 41.0 kDa |
| 39 | Pyruvate dehygrogenase subunit C | O70056 | |
| | (pyruvate dehydrogenase E1 | (Q9JU08) | |
| | component) | | |
| 43 | Dihydrolipoamide acetyltransferase | Q9JU07 | |
| | (pyruvate dehydrogenase component) | | |
| 47 | Alanyl - tRNA synthetase | Q9JYG6 | 100.4 kDa |
| 51 | Na+ translocating NADH quinone | Q9JVQ0 | 33.2 kDa |
| | reductase subunit C | | |
| 55 | Na+ translocating NADH quinone | Q9JVQ1 | 16.7 kDa |
| | reductase subunit D | | |
| 59 | Putative hydrolase | Q9JWG2 | 19.4 kDa |
| 63 | Hemagglutinin/hemolysin related | Q9JY30 | 119.1 kDa |
| | protein | | |
| 67 | Nitrogen assimilation regulatory | Q9K1K2 | 41.3 kDa |
| | protein (NtrX) | | |
| 71 | DNA processing chain A | Q9K1K1 | 50.8 kDa |
| 75 | Hypothetical protein | Q9JR28 | |
| 79 | BirA protein/Bvg accessory factor | Q9JXF1 | |
| 83 | Putative periplasmic protein | Q9JXF2 | |
| 87 | Cytochrome C precursor | Q9JWG4 | 16.7 kDa |
| 91 | Hypothetical protein | Q9JXH2 | |
| 95 | HemK protein | Q9JWH6 | 29.5 kDa |
| 99 | Probable ATP dependent helicase | Q9K181 | 86.1 kDa |
| | DinG | | |

*homologue codes can be used to retrieve protein information at http://www.expasy.org

### Example 2

### Mass Spectrometry Screen

Surface Enhanced Laser Desorption Ionisation Methods

1 Production of Rabbit Sera

Rabbits were immunised s.c with 60 μg N. lactamica protein pools (described previously) in 2 ml of 25% (v/v) alhydrogel administered over four sites. Primary vaccinations were administered to rabbits on day 1 of the experiment. The vaccinations were boosted on days 21 and 28 and challenge was on day 35 of the experiment.

1.1 Purification of IgG from Serum

A Protein G Sepharose Fast Flow gel column was packed as described by the manufacturer. The serum sample was diluted 1:4 in 20 mM sodium phosphate buffer, pH7.0 (buffer A) and the column equilibrated with the same buffer. The diluted sample was loaded onto the column and washed through the column with buffer A at a rate of 1 ml min⁻¹. The eluate was collected as 5 ml fractions. After 48 ml had washed through the column the buffer was changed for 0.1 M glycine, pH2.8 (buffer B). This was eluted through the column at the same rate as previously stripping the column of bound IgG. 22 ml of buffer B was washed through the column and the sample was collected in 2 ml fractions into 0.5 ml Tris, pH 9.0 to neutralise the pH.

2 Preparation of Detergent Extracted OMPs

A 500 ml broth culture of N. meningitidis, strain MC58 cap⁻, was centrifuged at 200 g for 60 min. The supernatant was discarded and the pellet washed with 100 ml PBS by centrifugation at 200 g for 30 min. The supernatant was again discarded and 2 ml PBS containing 0.3% (v/v) elugent was added for each gram of pellet. The pellet was homogenised and incubated at 37° C. with shaking for 20 min. The solution was centrifuged at 14,000 rpm for 10 min and the pellet discarded. To the supernatant 10 mM (w/v) EDTA, 0.5% (w/v) N-lauryl-sarcosine (Sigma) and 0.1% (v/v) of a 10% (w/v) PMSF solution was added. The supernatant containing extracted OMPs was stored at −20° C.

2.1 Separation of Detergent Extract by Preparative Electrophoresis

Preparative electrophoresis was carried out using the model 419 Prep-Cell (BioRad) as described in the protocol supplied. The gels used were non-denaturing and a gel consisting of 7% (v/v) protogel was used for separation of OMPs of <100 kDa.

3 Coating of Dynabeads with N. lactamica IgG and N. meningitidis Protein Pools

250μl tosylactivated dynabeads were placed into a 1.5 ml tube and the beads retained by magnet. The solution was removed and the beads were resuspended with mixing for 2 min in 250 μl 0.1 M borate buffer (pH 9.5). This was repeated twice. The buffer was then removed and the beads resuspended in 500 μl containing 30 μIgG. The beads were incubated for 18 hours at 37° C. with slow tilt rotation. The beads were blocked by resuspending them in 500 μl PBS containing 0.1% (w/v) BSA. This was repeated twice. The solution was removed and replaced with 0.2M Tris (pH8.5) containing 0.1% (w/v) BSA and incubated for 4 hours at 37° C. with slow tilt rotation. The solution was removed and the beads resuspended in 500 μl PBS containing 0.1% BSA. The beads were washed in 500 μl PBS containing 0.5% (v/v) Triton X100, resuspended in 500 μl PBS containing 0.1% BSA and finally resuspended in 100 μl PBS containing 0.1% BSA. 10 μl of the IgG coated bead solution was incubated with 50 μl N. meningitidis OMP pool for 4 hours with slow tilt rotation. The supernatant was removed and the beads washed with sterile water for 5 min in triplicate. Washed beads were resuspended in 10 μl sterile water for analysis. Beads coated with normal rabbit IgG and incubated with N. meningitidis OMPs were used as controls.

4 Analysis of Beads

2 μl beads were placed onto 1 μl 50% (v/v) acetonitrile on spot of H4 chips. These were left to dry and covered with 0.7 μl of a 10 mg/ml solution of sinapinic acid in 0.25% (v/v) trifluoric acid and 50% (v/v) acetonitile. ProteinChips (Ciphergen™) were read using the Ciphergen SELDI apparatus and accurate molecular weights of the proteins bound by the rabbit IgG were determined Calibration of the SELDI apparatus was carried out as described in the Ciphergen™ handbook.

The putative meningococcal proteins cross-reacting with IgG from N. lactamica antisera as identified in the screen were correlated to the N. meningitidis Group B genome database using ExPasy Tagident software. All proteins identified from the N. meningitidis genome have a molecular weight within 5% of that determined for the particular polypeptide in the SELDI screen. Preferred proteins have molecular weights within 2% of the molecular weight for the SELDI identified polypeptide.

N. meningitidis nucleic acid sequences identified in the mass spectrometry screen as encoding candidate vaccine antigens and their translation products are shown in SEQ ID NOS: 103-199, and are set out in Table 2 below.

TABLE 2
(proteins in bold show only 2% difference in molecular weight from SELDI
identified polypeptide; other proteins are within 5% of the SELDI identified polypeptide)

| SEQ | | | | |
|---|---|---|---|---|
| ID | SELDI | | Protein | Locus in |
| NO. | mw | Putative N. meningitidis protein | mw | genome |
| 103 | 11226.0 Da | FK506 Binding Protein | 11788.52 Da | NMB0027 |
| 105 | | Glutamyl T-RNA amidotransferase subunit C | 10958.36 Da | NMB1355 |
| 107 | | Aspartate 1-decarboxylase | 11145.70 Da | NMB1282 |
| 109 | | Hypothetical protein | 11411.19 Da | NMB0837 |
| 111 | 13712.8 Da | Probable glycine cleavage system H | 13643.07 Da | NMB0575 |
| | | protein | | |
| 113 | | 50S ribosomal protein L19 | 13767.95 Da | NMB0589 |
| 115 | | 50S ribosomal protein L20 | 13710.15 Da | NMB0723 |
| 117 | | Ribonuclease P protein component | 14211.41 Da | NMB1905 |
| 119 | | 30S ribosomal protein S6 | 13949.02 Da | NMB1323 |
| 121 | 17378.9 Da | Bacterioferritin A | 17961.25 Da | NMB1207 |
| 123 | | Disulphide bond formation protein B | 17701.21 Da | NMB1649 |
| 125 | | Dihydrofolate reductase | 17751.52 Da | NMB0308 |
| 127 | | (3R)-hydroxymyristoyl-[acyl carrier protein] | 16626.53 Da | NMB0179 |
| | | dehydratase | | |
| 129 | | Fimbral Protein (Pilin) | 17298.65 Da | NMB0018 |
| | | H8 Outer Membrane Protein | 16885.79 Da | NMB1533 |
| 131 | | 2C-methyl-D-erythritol 2,4-cyclodiphosohate | 17019.54 Da | NMB1512 |
| | | synthase | | |
| | | Superoxide Dismutase [Cn-Zn] | 17360.36 Da | NMB1398 |
| 133 | | Hypothetical protein | 17284.82 Da | NMB1816 |
| 135 | 26868.0 Da | 3-dehydroquinate dehydratase | 27186.21 Da | NMB1446 |
| 137 | | Acyl-[acyl-carrier protein]-UDP-N- | 28154.91 Da | NMB0178 |
| | | acetylglucosamine O-acetyltransferase | | |
| 139 | | Probable septum site-determining protein | 26221.11 Da | NMB0170 |
| 141 | | Hypothetical methyltransferase | 27037.62 Da | NMB1328 |
| 145 | | Na-translocating NADH-quinone reductase | 27606.57 Da | NMB0567 |
| | | subunit C | | |
| 147 | | 3-methyl-2-oxobutanoate | 27739.25 Da | NMB0870 |
| | | hydroxymethyletransferase | | |
| 149 | | Pyridoxal phosphate biosynthetic protein | 26565.58 Da | NMB0448 |
| 151 | | 1-acyl-sn-glycerol-3-phosphate | 27943.25 Da | NMB1294 |
| | | acetyltransferase | | |
| 153 | | Thiazole biosynthesis protein | 28067.06 Da | NMB2071 |
| 155 | | 3-demethylubiquinone-9 3- | 26529.48 Da | NMB2030 |
| | | metyltransferase | | |
| 157 | | Hypothetical protein | 27417.04 Da | NMB2054 |
| | 28173.6 Da | 3-dehydroquinate dehydratase | 27186.21 Da | NMB1446 |
| 159 | | Shikimate 5-Dehydrogenase | 28564.71 Da | NMB0358 |
| 161 | | Competence lipoprotein comL | 29274.90 Da | NMB0703 |
| 163 | | Dihydrolipocolinate reductase | 28328.10 Da | NMB0203 |
| | | Acyl-[acyl-carrier protein]-UDP-N- | 28154.91 Da | NMB0178 |
| | | acetylglucosamine O-acetyltransferase | | |
| | | Hypothetical methyltransferase | 27037.62 Da | NMB1328 |
| | | Na-translocating NADH-quinone reductase | 27606.57 Da | NMB0567 |
| | | subunit C | | |
| | | 3-methyl-2-oxobutanoate | 27739.25 Da | NMB0870 |
| | | hydroxymethyletransferase | | |
| | | 1-acyl-sn-glycerol-3-phosphate | 27943.25 Da | NMB1294 |
| | | acetyltransferase | | |
| | | Thiazole biosynthesis protein | 28067.06 Da | NMB2071 |
| 165 | | TonB protein | 29198.89 Da | NMB1730 |
| | | Hypothetical protein | 27417.04 Da | NMB2054 |
| 167 | 33719.3 a | GTP binding protein | 34617.03 Da | NMB0678 |
| 169 | | Glycerol-3-phosphate dehydrogenase | 35337.90 Da | NMB2060 |
| 171 | | Porphobilinogen deaminase | 33478.47 Da | NMB0539 |
| 173 | | 33 kDa chaperonin | 33204.66 Da | NMB2000 |
| 175 | | Lacto-N-neotetraose biosynthesis glycosyl | 32790.00 Da | NMB1926 |
| | | transferase | | |
| 177 | | UDP-3-O-[3-hydroxymyristoyl]- | 33986.69 Da | NMB0017 |
| | | acetylglucosamine deacetylase | | |
| 179 | | T-RNA delta(2)-isopentenylpyrophosphate | 34870.26 Da | NMB0935 |
| | | transferase | | |
| | | Class 3 protein, porin | 33845.23 Da | PorB |
| | | Class 3 protein, porin | 33845.26 Da | PorB |
| | | Class 3 protein, porin | 33868.36 Da | PorB |
| | | Class 3 protein, porin | 33786.29 Da | NMB2039 |
| 181 | | Proline iminopeptidase | 34956.75 Da | NMB0927 |
| 183 | | Recombination associated protein | 33263.72 Da | NMB0851 |
| 185 | | Glucose-1-phosphate thymidylyltransferase | 32161.71 Da | NMB0062 |
| | | Transposase for insertion sequence element | 32758.54 Da | |
| | | IS1106 | | |
| 187 | | T-RNA psuedouridine synthase B | 33632.50 Da | NMB1374 |
| 189 | | Hypothetical adenine-specific methylase | 33955.36 Da | NMB1655 |
| 191 | 66656.4 Da | Chaperone protein DNA K (heat shock | 68791.66 Da | NMB0554 |
| | | protein 70) | | |
| 193 | | 1-deoxy-D-xylulose 5-phosphate synthetase | 68749.57 Da | NMB1876 |
| 195 | | DNA primase | 65915.11 Da | NMB1537 |
| 198 | | Glutaminyl-T-RNA synthetase | 64649.85 Da | NMB1560 |
| | | Transferrin binding protein B | 63343.98 Da | TbpB |

It interesting to note that this method also identifies a number of proteins that are known to be strong vaccine antigens, such as TbpB, class 3 protein, H8 outer membrane protein and Cu,Zn superoxide dismutase. This clearly validates the effectiveness of the present method.

The identified sequences were further analysed to determine whether any of the putative proteins comprised signal sequences or transmembrane domains. Presence of these distinctive motifs is often indicative of surface exposure and can help to further identify suitable vaccine antigens.

Th presence of signal peptides was determined using the SignalP algorithm, and for transmembrane domains the TMpred algorithm was used. Both algorithms are commonly known in the art and are available from www.expasy.org (The EXPASy™, Expert Protein Analysis System is hosted by the proteomics server of the Swiss Institute of Bioinformatics)

The results of the SignalP and TMpred analysis is shown in Table 3.

TABLE 3

| | | | Signal |
|---|---|---|---|
| SEQ ID | | TM domain? | Sequence? |
| NO. | Putative N. meningitidis protein | (X/3) (TMpred) | (X/3) (SignalP) |
| 103 | FK506 Binding Protein | X | X |
| 105 | Glutamyl T-RNA amidotransferase subunit C | X | X |
| 107 | Aspartate 1-decarboxylase precursor | X | X |
| 109 | Hypothetical protein | X | X |
| 111 | Probable glycine cleavage system H protein | X | X |
| 113 | 50S ribosomal protein L19 | X | X |
| 115 | 50S ribosomal protein L20 | X | X |
| 117 | Ribonuclease P protein component | X | X |
| 119 | 30S ribosomal protein S6 | X | X |
| 121 | Bacterioferritin A | X | X |
| 123 | Disulphide bond formation protein B | 3 | 3 |
| 125 | Dihydrofolate reductase | 3 | 3 |
| 127 | (3R)-hydroxymyristoyl-[acyle carrier protein] | 3 | X |
| | dehydratase | | |
| 129 | Fimbral Protein Precursor (Pilin) | 3 | X |
| | H8 Outer Membrane Protein Precursor | 3 | 3 |
| 131 | 2C-methyl-D-erythritol 2,4-cyclodiphosphate | 3 | X |
| | synthase | | |
| | Superoxide Dismutase [Cn-Zn] Precursor | 3 | 3 |
| 133 | Hypothetical protein | X | X |
| 135 | 3-dehydroquinate dehydratase | 3 | X |
| 137 | Acyl-[acyl-carrier protein]-UDP-N- | 3 | X |
| | acetylglucosamine O-acetyltransferase | | |
| 139 | Probable septum site-determining protein | X | X |
| 141 | Hypothetical methyltransferase | X | 3 |
| 145 | Na-translocating NADH-quinone reductase | 3 | X |
| | subunit C | | |
| 147 | 3-methyl-2-oxobutanoate | X | X |
| | hydroxymethyletransferase | | |
| 149 | Pyridoxal phosphate biosynthetic protein | X | X |
| 151 | 1-acyl-sn-glycerol-3-phosphate | 3 | X |
| | acetyltransferase | | |
| 153 | Thiazole biosynthesis protein | 3 | X |
| 155 | 3-demethylubiquinone-9 3-metyltransferase | X | X |
| 157 | Hypothetical protein | X | X |
| | 3-dehydroquinate dehydratase | 3 | X |
| 159 | Shikimate 5-Dehydrogenase | 3 | X |
| 161 | Competence lipoprotein comL precursor | 3 | 3 |
| 163 | Dihydrolipocolinate reductase | 3 | X |
| | Acyl-[acyl-carrier protein]-UDP-N- | 3 | X |
| | acetylglucosamine O-acetyltransferase | | |
| | Hypothetical methyltransferase | X | X |
| | Na-translocating NADH-quinone reductase | 3 | 3 |
| | subunit C | | |
| | 3-methyl-2-oxobutanoate | 3 | X |
| | hydroxymethyletransferase | | |
| | 1-acyl-sn-glycerol-3-phosphate | 3 | X |
| | acetyltransferase | | |
| | Thiazole biosynthesis protein | 3 | X |
| 165 | TonB protein | 3 | 3 |
| | Hypothetical protein | X | X |
| 167 | GTP binding protein | X | 3 |
| 169 | Glycerol-3-phosphate dehydrogenase | 3 | X |
| 171 | Porphobilinogen deaminase | X | X |
| 173 | 33 kDa chaperonin | X | X |
| 175 | Lacto-N-neotetraose biosynthesis glycosyl | X | X |
| | transferase | | |
| 177 | UDP-3-O-[3-hydroxymyristoyl]- | X | X |
| | acetylglucosamine deacetylase | | |
| 179 | T-RNA delta(2)-isopentenylpyrophosphate | 3 | X |
| | transferase | | |
| | Class 3 protein, porin | 3 | 3 |
| | Class 3 protein, porin | 3 | 3 |
| | Class 3 protein, porin | 3 | 3 |
| | Class 3 protein, porin | 3 | 3 |
| 181 | Proline iminopeptidase | 3 | X |
| 183 | Recombination associated protein | X | X |
| 185 | Glucose-1-phosphate thymidylyltransferase | 3 | X |
| | Transposase for insertion sequence element | | |
| | IS1106 | | |
| 187 | T-RNA psuedouridine synthase B | X | X |
| 189 | Hypothetical adenine-specific methylase | 3 | X |
| 191 | Chaperone protein DNA K (heat shock protein | X | X |
| | 70) | | |
| 193 | 1-deoxy-D-xylulose 5-phosphate synthetase | 3 | X |
| 195 | DNA primase | 3 | X |
| 198 | Glutaminyl-T-RNA synthetase | X | X |
| | Transferrin binding protein B | 3 | 3 |

Although a number of the identified sequences appear to contain putative signal sequences or transmembrane domains it is surprising that a significant number also do not.

Hence, it can be seen that the methods of the invention provide methods for identifying polypeptides not previously known to have antigenic potential.

1. A method for identifying an antigen comprising:
a. obtaining antibodies raised against a commensal Neisseria or an extract from a commensal Neisseria;
b. contacting the antibodies with one or more polypeptides obtained from an expression library of either a commensal bacteria or a pathogenic bacteria;
c. determining whether one or more polypeptides bind to one or more of said antibodies;
d. where a polypeptide bind to an antibody, identifying that polypeptide as an antigen; and
e. isolating a clone that expresses the antigen from the expression library.

2. A method according to claim 1, wherein step (e) comprises:
i. identifying the molecular weight of the polypeptide that bind to the antibody;
ii. correlating the molecular weight with the molecular weight of polypeptide encoded by the genome of the bacteria from which the polypeptide is derived; and
iii. determining an identity for the polypeptide and the corresponding nucleic acid that encodes the polypeptide.

3. A method according to claim 2, wherein the molecular weight of the polypeptide is determined via mass spectrometry, electrophoresis or chromatography.

4. A method according to claim 1, wherein the polypeptides of step (b) are presented in the form of a phage display library, and in step (e) the clone that expresses the polypeptide antigen is located within a phagemid vector.

5. A method according to claim 4, wherein the phage display library is in lamda phage.

6. A method according to claim 1, wherein the expression library is derived from a pathogenic bacterial genome.

7. A method according to claim 1, wherein the expression library is derived from a commensal bacterial genome.

8. A method according to claim 7, further comprising the steps of:
i. using the nucleic acid sequence of the isolated clone encoding the polypeptide antigen from the commensal bacteria to identify homologous sequences in pathogenic bacteria; and
ii. cloning the homologous sequences from the pathogenic bacteria in order to generate the equivalent pathogenic bacterial polypeptide antigen.

9. A method according to claim 1, wherein the commensal Neisseria is selected from the group consisting of N lactamica; N. cinerea; N. sicca; N. subflava; N. elogata; N. flavescens; N. perflava and N polysaccharea.

10. A method according to claim 1, wherein the pathogenic bacteria is selected from the Neisseriaceae/Pasteurellaceae family of Gram negative bacteria.

11. A method according to claim 10, wherein the pathogenic bacteria is N meningitidis.

12. A method according to claim 1, wherein the antibodies are raised against whole commensal Neisseria cells.

13. A method according to claim 1, wherein the antibodies are raised against a protein extract from commensal Neisseria cells.

14. A method according to claim 13, wherein the protein extract is an outer membrane protein extract.

15. A method according to claim 1, wherein the antibodies are purified so as to be enriched for IgG.

16. A method for identifying an antigen, suitable for inclusion in a vaccine composition, comprising the steps of:
a. obtaining sera raised against an outer membrane protein extract of N lactamica genome;
b. contacting the sera with a phage display library comprising the entire N. lactamica genome;
c. identifying a phage that tests positive for a binding interaction with the sera, and isolating the positive phage;
d. extracting phagemid vector from the positive phage and characterising the cloned N lactamica genomic sequence located therein; and
e. determining the polypeptide encoded by the N lactamica genomic sequence and identifying the polypeptide as an antigen.

17. A method according to claim 16, further comprising the step of:
f. comparing the sequence of the N. lactamica polypeptide antigen with a N. meningitidis genomic library in order to identify the N meningitidis homologue polypeptide antigen.

18. A method for identifying an antigen, suitable for inclusion in a vaccine composition, comprising the steps of:
a. obtaining sera raised against an outer membrane protein extract of N lactamica;
b. isolating the IgG component of the sera;
C. binding the isolated IgG to a solid phase;
d. contacting the bound IgG with polypeptides obtained from an extract of N meniningitidis cells;
e. isolating solid phase-IgG-polypeptide complexes that are formed by the binding of polypeptides to IgG;
f. analysing solid phase-IgG-polypeptide complexes via SELDI mass spectrometry;
g. correlating molecular weights obtained for the polypeptide from (f) with molecular weights of known and putative polypeptides from the N. meningitidis genome database; and
h. identifying as antigens those N meningitidis polypeptides encoded by genes determined from the correlated molecular weights of (g).

19. A method of preparing a vaccine consisting of the steps of identifying an antigen according to the method of claim 1, and combining the antigen with a pharmaceutically acceptable carrier.

20. A method for preparing a vaccine composition consisting of the steps of:
a. obtaining sera raised against a commensal bacteria, or an extract from a commensal Neisseria;
b. contacting the sera with one or more polypeptides obtained from either a commensal or a pathogenic bacteria;
c. determining whether the one or more polypeptides bind to antibodies present in the sera;
d. where a polypeptide binds to an antibody in the sera, identifying that polypeptide as an antigen; and
e. combining the antigen with a pharmaceutically acceptable carrier.

21. A method for preparing a vaccine composition consisting of the steps of:
a. obtaining sera raised against a commensal Neisseria , or an extract from a commensal Neisseria;
b. contacting the sera with one or more polypeptides obtained from either a commensal or a pathogenic bacteria;
c. determining whether the one or more polypeptides bind to antibodies present in the sera;
d. where a polypeptide binds to an antibody in the sera, identifying that polypeptide as an antigen;
e. obtaining the nucleic acid sequence that encodes the antigen; and
f. preparing a vaccine composition comprising the nucleic acid sequence and a pharmaceutically acceptable carrier.

22. (cancelled)

23. An antigen identified by the method of claim 1.

24. A polypeptide encoded by all or a part of a nucleic acid sequence selected from the group consisting of SEQ ID NOS: 1, 5, 11, 15, 19, 23, 27, 31, 35, 39, 43, 47, 51, 55, 59, 63, 67, 71, 75, 79, 83, 87, 91, 95, and 99.

25. A polypeptide antigen expressed from all or part of a nucleic acid sequence selected from the group consisting of SEQ ID NOS: 1, 5, 11, 15, 19, 23, 27, 31, 35, 39, 43, 47, 51, 55, 59, 63, 67, 71, 75, 79, 83, 87, 91, 95, and 99.

26. A polypeptide antigen expressed from a nucleic acid sequence having at least 90% homology with a nucleic acid sequence selected from the group consisting of SEQ ID NOS: 1, 5, 11, 15, 19, 23, 27, 31, 35, 39, 43, 47, 51, 55, 59, 63, 67, 71, 75, 79, 83, 87, 91, 95, and 99.

27. An isolated nucleic acid molecule selected from the group consisting of SEQ ID NOS: 1, 5, 11, 15, 19, 23, 27, 31, 35, 39, 43, 47, 51, 55, 59, 63, 67, 71, 75, 79, 83, 87, 91, 95, and99.

28. A vector comprising one or more nucleic acid sequences selected from the group consisting of SEQ ID NOS: 1, 5, 11, 15, 19, 23, 27, 31, 35, 39, 43, 47, 51, 55, 59, 63, 67, 71, 75, 79, 83, 87, 91, 95, and 99.

29. A polypeptide selected from the group consisting of SEQ ID NOS: 2, 6-8, 12, 16, 20, 24, 28, 32, 36, 40, 44, 48, 52, 56, 60, 64, 68, 72, 76, 80, 84, 88, 92 and 100.

30. A vaccine composition comprising:
a. apolypeptide expressed from all or part of a nucleic acid sequence selected from the group consisting of SEQ ID NOS: 1, 5, 11, 15, 19, 23, 27, 31, 35, 39, 43, 47, 51, 55, 59, 63, 67, 71, 75, 79, 83, 87, 91, 95, and 99; and
b. a pharmaceutically acceptable carrier.

31. A vaccine composition comprising:
a. apolypeptide expressed from all or part of a nucleic acid sequence selected from the group consisting of SEQ ID NOS: 3, 9, 13, 17, 21, 25, 29, 33, 37, 41, 45, 49, 53, 57, 61, 65, 69, 73, 77, 81, 85, 89, 93, 101, 103, 105, 107, 109, 111, 113, 115, 117, 119, 121, 123, 125, 127, 129, 131, 133, 135, 137, 139, 141, 145, 147, 149, 151, 153, 155, 157, 159, 161, 163, 165, 167, 169, 171, 173, 175, 177, 179, 181, 183, 185, 187, 189, 191, 193, 195, and 198; and
b. a pharmaceutically acceptable carrier.

32. A composition according to any one of claims 30 and 31, wherein the polypeptide is expressed from all or part of a nucleic acid sequence having at least 90% homology to one or more of the sequences recited in claim 33.

33. A vaccine composition comprising:
a. all or a part of a polypeptide selected from the group consisting of SEQ ID NOS: 2, 6-8, 12, 16, 20, 24, 28, 32, 36, 40, 44, 48, 52, 56, 60, 64, 68, 72, 76, 80, 84, 88, 92, and 100; and
b. a pharmaceutically acceptable carrier.

34. A vaccine composition comprising:
a. all or a part of a polypeptide selected from the group consisting of SEQ ID NOS: 4, 10, 14, 18, 22, 26, 30, 34, 38, 42, 46, 50, 54, 58, 62, 66, 70, 74, 78, 82, 86, 90, 94, 102, 104, 106, 108, 110, 112, 114, 116, 118, 120, 122, 124, 126, 128, 130, 132, 134, 136, 138, 140, 142-144, 146, 148, 150, 152, 154, 156, 158, 160, 162, 164, 166, 168, 170, 172, 174, 176, 178, 180, 182, 184, 186, 188, 190, 192, 194, 196-197, and 199, and
b. a pharmaceutically acceptable carrier.

35. (cancelled)

36. (cancelled)

37. (cancelled)

38. A method of preparing a composition for vaccination against infection by pathogenic bacteria, comprising:
a. obtaining a first antigen from a commensal Neisseria;
b. comparing (i) the amino acid sequence of the first antigen with the amino acid sequence of a second antigen from a pathogenic bacteria, or (ii) comparing the sequence of a nucleic acid which codes for the first antigen with the sequence of a nucleic acid that codes for the second antigen; and, if the first antigen is homologous to the second antigen or if the nucleic acid sequence for the first antigen is homologous to the nucleic acid sequence for the second antigen; and
c. preparing a composition for vaccination against bacterial infection comprising the first antigen.

39. A method according to claim 38, wherein the second antigen is derived from a library of antigens from a pathogenic bacteria or the nucleic acid sequence coding for the second antigen is derived from a library of nucleic acid sequences coding for antigens from a pathogenic bacteria.

40. A method according to claim 38, wherein the commensal nucleic acid sequence is compared with a genome sequence of a pathogenic Neisseria.

41. A vaccine composition according to claim 30, further comprising neisserial outer membrane vesicles (OMVs).

42. An antibody that binds to an antigen according to claim 23.

43. An antibody that binds to a polypeptide antigen of claim 24.

44. A pharmaceutical composition comprising an antibody of claim 42.

45. A method of preparing a vaccine consisting of the steps of identifying an antigen according to the method of claim 16, and combining the antigen with a pharmaceutically acceptable carrier.

46. A method of preparing a vaccine consisting of the steps of identifying an antigen according to the method of claim 18, and combining the antigen with a pharmaceutically acceptable carrier.

47. A composition according to any one of claims 30 and 31, wherein the polypeptide is expressed from all or part of a nucleic acid sequence having at least 90% homology to one or more of the sequences recited in claim 34.
